(19) United States
(12) Patent Application Publication
(10) Pub. No.: US 2026/0215727 A1
(43) Pub. Date: Jul. 30, 2026

(54) ULTRA-WIDEBAND AND MULTI-ANGLE MICROWAVE IMAGING FOR EARLY DETECTION OF BREAST CANCER

(71) Applicants: BREAST CANCER EM SYSTEMS (BCEMS) LTD., Gvat (IL); DETECTION AND PROTECTION SYSTEMS (DPES) LTD., Beer Sheva (IL)

(72) Inventors: Nathan BLAUNSTEIN, Beer Sheva (IL); Sergey SHIPILOV, Seversk (RU); Rail SATAROV, Tomsk (RU); Alexander EREMEEV, Tomsk (RU); Ilya TSEPLYAEV, Tomsk (RU)

(21) Appl. No.: 19/143,533

(22) PCT Filed: Dec. 26, 2023
(86) PCT No.: PCT/IB2023/063245

§ 371 Date: Jun. 26, 2025

Related U.S. Application Data
(60) Provisional application No. 63/435,291, filed on Dec. 26, 2022.

(51) Int. Cl. A61B 5/00; A61B 5/0507; A61B 90/00

(52) U.S. Cl. CPC A61B 5/4312; A61B 5/0507; A61B 5/704; A61B 5/708; A61B 90/36

(57) ABSTRACT

An apparatus and method for early detection of breast cancer uses ultra-wideband and multi-angle microwave imaging. The apparatus includes a matching dielectric layer and a conformal antenna array with antenna elements arranged in a hemispherical pattern. The method achieves high resolution and high signal-to-clutter ratio using synthesized focusing (SF) and specialized clutter rejection (SCR) algorithms. The improved performance of the method is demonstrated experimentally using a realistic, heterogeneous breast phantom composed of materials whose dielectric properties closely match those of skin, adipose, glandular, and tumorous breast tissue.

### CROSS-REFERENCE TO RELATED APPLICATIONS

This application is related to and claims priority from commonly owned U.S. Provisional Patent Application No. 63/435,291, entitled “Method of Electromagnetic Tomography and Radio Imager for Diagnostics of the Breast Cancer”, filed on Dec. 26, 2022, the disclosure of which is incorporated by reference in its entirety herein.

### TECHNICAL FIELD

The present invention relates to microwave imaging and specifically to apparatus and methods for early detection of breast cancer using ultra-wideband (UWB) and multi-angle microwave imaging.

### BACKGROUND OF THE INVENTION

In screening for breast cancer, the use of non-ionizing microwave radiation for mammographic imaging may provide significant benefits in terms of diagnostic specificity and sensitivity and specificity, spatial resolution, and patient comfort over current methods that employ ionizing X-ray tomography or ultrasonic scanning. The microwave frequency range proves to be a good balance between penetration depth into breast tissue, and spatial resolution for tumor detection and imaging.

A technical paper by M. D. Lazebnik et al., entitled “A large-scale study of the ultrawideband microwave dielectric properties of normal, benign and malignant breast tissues obtained from cancer surgeries,” appearing in Physics in Medicine and Biology, Vol. 52, pp. 6093-6115, 2007 (hereinafter “Lazebnik”), presents extensive measurements of the microwave dielectric properties of a variety of normal, malignant and benign breast tissues measured over a frequency range of 0.5 to 20 gigahertz (GHz) using a precision open-ended coaxial probe. The contrast in dielectric properties between malignant and normal adipose-dominated tissues in the breast is found to be as large as 10:1; whereas the contrast between malignant and normal glandular/fibroconnective tissues in the breast is no more than about 10%.

International patent application no. WO2023014320A1, to I. Akduman et al., entitled “A Microwave Breast Cancer Screening System,” and dated Feb. 9, 2023, discloses a microwave-based breast cancer screening and early diagnostics imaging system which uses mild compression to electromagnetically homogenize the heterogeneous breast media or, to some degree, decrease the breast dimension to 2D. The device is capable of providing multi-angle examination, if necessary, and produces horizontal and vertical cross-sectional images based on the polarization of the antennas used for scanning. Merging two-sectional images can give a possibility of 3D microwave representation of the breast tissue that allows identification of the malignant/cancerous/harmful tissues/cells through cross validation.

One of the problems that has plagued 3D microwave imaging is the heavy computational burden involved in solving a fully three-dimensional (3D) inverse-scattering problem for the dielectric properties of a sample volume under test. International Publication Number WO2013/012631 A1, to T. M. Grzegorczyk, entitled “Fast Tomographic Microwave Imaging”, teaches microwave imaging equipment utilizing an array of antennas operated to collect electromagnetic field information for a material being imaged. Image processing method and apparatus use the discrete dipole approximation (DDA) and drastically reduce the time required to process the measured data and estimate the properties of the interrogated material.

Both DDA and the Born scattering approximation are widely used in simulation tools that compute scattering and absorption by targets of arbitrary geometry. However, such approximations are known to be highly inaccurate for a dielectric material with significant inhomogeneities, such as breast tissue, in which dispersion and diffraction effects play a significant role.

### SUMMARY OF THE INVENTION

The invention discloses apparatus and methods for UWB and multi-angle microwave imaging of breast tissue, for early detection of breast cancer.

According to one aspect of the presently disclosed subject matter, there is provided an apparatus for forming a microwave image for the purpose of detection of breast cancer in a patient. The apparatus includes: a matching dielectric layer having a first surface proximal to a breast of the patient and a second surface proximal to a conformal antenna array comprising a multiplicity of antenna elements; an ultra-wideband (UWB) radar signal generator and an UWB radar receiver which are in electrical communication with the conformal antenna array; a digital image processor in electrical communication with the radar receiver; and an output device for displaying image data provided by the digital image processor. Each antenna element of the conformal antenna array is configured to transmit and/or to receive microwave signals over a predetermined range of microwave frequencies.

According to some aspects, the UWB radar signal generator and/or the UWB radar receiver include(s) a vector network analyzer (VNA).

According to some aspects, the UWB radar signal generator generates pulsed signals having linear or stepped frequency modulation.

According to some aspects, the UWB radar receiver performs frequency to time delay pre-processing.

According to some aspects, the digital image processor performs a synthesized focusing (SF) algorithm and/or a specialized clutter rejection algorithm (SCR).

According to some aspects, the multiplicity of antenna elements is distributed over a multiplicity of azimuth and elevation angles in a hemispherical pattern.

According to some aspects, the conformal antenna array comprises multiple-input, multiple-output (MIMO) electronic channel switching.

According to some aspects, the multiplicity of antenna elements comprises at least 96 elements.

According to some aspects, a pair of antenna elements, in which one element transmits and the other receives, is activated at a given time.

According to some aspects, the matching dielectric layer is configured to improve penetration of the microwave signals into the breast of the patient.

According to some aspects, the predetermined range of microwave frequencies includes a range of two to eight gigahertz.

According to some aspects, the output device is configured to display three-dimensional images and/or two-dimensional cross-sectional images.

According to some aspects, the apparatus also includes an examination table supporting the patient in a prone position, the table having a cutout to allow the breast of the patient to protrude into a space beneath the table, under a force of gravity.

According to another aspect of the presently disclosed subject matter, there is provided a computer usable non-transitory storage medium having computer-executable instructions that perform a method of imaging a breast of a patient from microwave measurement data. The method includes: transmitting ultra-wideband (UWB) microwave signals into the breast; receiving signals in a conformal antenna array; applying frequency-to-time delay pre-processing; applying a synthesized focusing (SF) algorithm; and applying a specialized clutter rejection (SCR) algorithm.

According to some aspects, the method also includes generating one or more output images for display.

According to some aspects, the output images include a coronal and/or a sagittal cross-sectional image.

According to some aspects, the UWB microwave signals propagate through a matching dielectric layer which is proximal to the breast and which is configured to improve penetration of the microwave signals into the breast.

According to some aspects, the conformal antenna array comprises a multiplicity of antenna elements distributed over a multiplicity of azimuth angles and a multiplicity of elevation angles in a hemispherical pattern.

According to some aspects, a total number of angular locations, defined as the product of a number of angles in the multiplicity of azimuth angles and a number of angles in the multiplicity of elevation angles, is between 800 and 1500.

According to some aspects, the frequency-to-time delay pre-processing is performed using a Fourier transform or a finite impulse response (FIR) filter.

According to some aspects, the SF algorithm comprises summation of a signals over a multiplicity of azimuth angles (φₙ) and elevation angles (θₘ), each signal characterized by a focusing time delay (τ_{m,n}).

According to some aspects, the SCR algorithm comprises subtraction of constant values (Q_{m, f(n)}) which may be different for different elevation angles (θₘ).

### BRIEF DESCRIPTION OF THE DRAWINGS

Some embodiments of the present invention are herein described, by way of example only, with reference to the accompanying drawings. With specific reference to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1: A block diagram of an apparatus for forming a 3D microwave image from UWB and multi-angle microwave measurement data, according to an embodiment of the invention.

FIG. 2: A perspective drawing of a conformal antenna array having a multiplicity of antenna elements distributed in a hemispherical pattern.

FIG. 3: A block diagram of an exemplary microwave imaging method, according to the invention.

FIG. 4: A cross-sectional diagram of a heterogeneous breast phantom used to evaluate the spatial resolution and performance of the microwave imaging method of the invention.

FIG. 5A: An exemplary coronal cross-sectional image of the heterogeneous breast phantom, obtained using the specialized clutter rejection algorithm of the invention.

FIG. 5B: An image analogous to FIG. 5A obtained using a standard clutter rejection algorithm.

FIG. 6A: An exemplary sagittal cross-sectional image of the heterogeneous breast phantom, obtained using the specialized clutter rejection algorithm of the invention.

FIG. 6B: An image analogous to FIG. 6A obtained using a standard clutter rejection algorithm.

FIG. 7: A graph showing the signal-to-clutter improvement achieved by using synthesized focusing instead of standard focusing.

### DETAILED DESCRIPTION OF THE INVENTION

The principles and operation of the present invention may be better understood with reference to the drawings and the accompanying description.

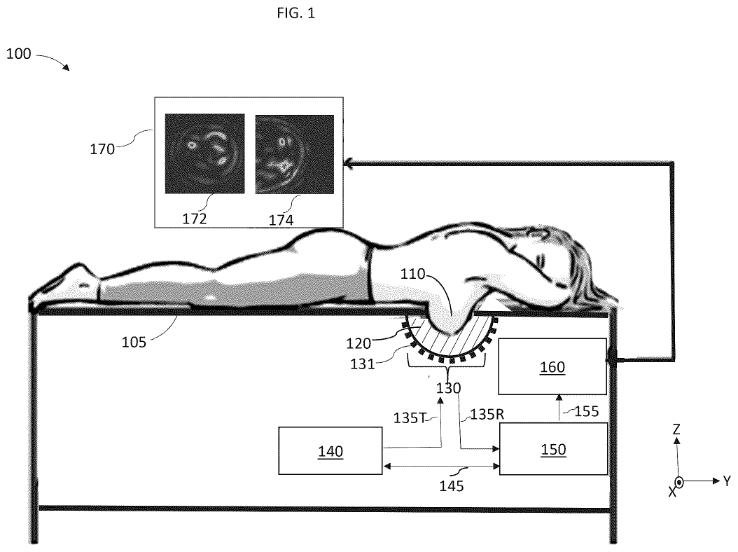

FIG. 1 shows a block diagram of an apparatus 100 for forming a 3D microwave image from UWB and multi-angle microwave measurement data, according to an embodiment of the invention. The patient lies prone on an examination table 105, which has a cutout to allow the breast 110 of the patient, i.e. the material under test, to protrude into a space beneath the table, under a force of gravity. As shown in FIG. 1, the X-Y plane is parallel to the surface of table 105, and the Z-axis points vertically upward.

A matching dielectric layer 120 is fitted to the breast from below. Layer 120 is comprised of a material with a relative dielectric constant that roughly matches that of normal breast tissue, at a given microwave frequency. For example, at 4 GHz, typical values of the relative dielectric constant for normal breast tissue are between five and ten. A suitable material for layer 120 may be made, for example, using a mixture of polyurethane and graphite powder comprising 90% to 95% polyurethane, by weight.

Surrounding layer 120 is a conformal antenna array 130, which includes a multiplicity of antenna elements 131 distributed in a hemispherical pattern. In practical use of the invention for mammographic screening, the patient must hold their breath during the time in which the breast tissue is irradiated, so as to reduce blurring of images caused by chest movement. The multiple-input, multiple-output (MIMO) transmission and reception provided by the multiplicity of antenna elements in array 130 enables a drastic reduction in the required irradiation time, as compared with existing tomographic devices that rely on scanning techniques. Typically, the multiplicity of antennas in array 130 may contain 96 antenna elements or more.

One or more elements of array 130 act as emitters and transmit UWB microwave signals that penetrate into breast tissue 110 to a depth of typically 5 to 10 centimeters. The transmitted signal waveforms are provided to the array 130 by the UWB radar signal generator 140 via path 135T.

In an exemplary embodiment of the invention, high spatial resolution is achieved using a transmitted signal consisting of short pulses with linear or stepped frequency modulation, such as that which is commonly found in a Vector Network Analyzer (VNA). For example, the transmission carrier frequency may increase in several incremental frequency steps from, say, 2 GHz to 8 GHz within each transmitted pulse. A typical half-power beamwidth for an individual antenna element may be 2 degrees in azimuth and elevation at 4 GHz.

At any given moment in time, each antenna element may be used as either a receiving or a transmitting antenna. When the same antenna element is used for both transmission and reception, the scattering geometry is monostatic; in all other cases the scattering geometry is bistatic or, more generally, multistatic. In some embodiments of the invention, a pair of antennas, in which one antenna element transmits and one receives, is activated at any given time. In this case, the scattering geometry at the given time is bistatic.

In order to increase spatial resolution, the effective beamwidth of a given antenna element may be reduced from its physical beamwidth through the use of synthetic aperture processing, a technique which is well known to those skilled in the art of radar signal processing. In this way, the spatial resolution may be improved to a few millimeters, which is crucial in enabling early detection of tumors.

The received signals propagate along path 135R into a UWB radar receiver 150. Receiver 150 also exchanges timing and synchronization signals with the UWB radar signal generator 140 via signal path 145. At the receiver 150, the received signal is converted in amplitude and phase from the frequency domain to the time-delay domain by means of Fourier transform processing. This may be implemented in a VNA or digitally using a digital Fourier transform (DFT) processor or a finite impulse response (FIR) filter.

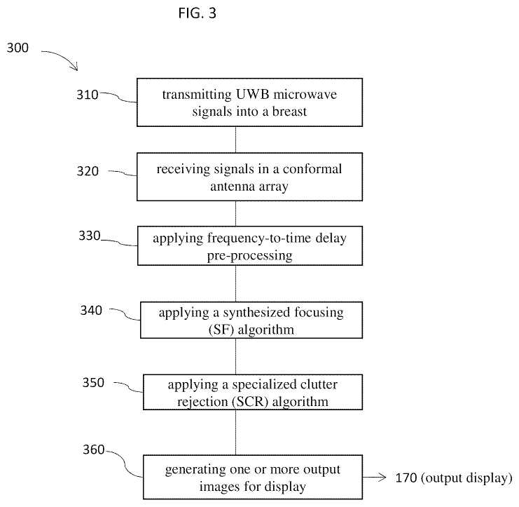

The time delay signals are time-sampled and sent via path 155 to a digital image processor 160, which executes a series of algorithms including synthesized focusing and specialized clutter-rejection, which are further described in connection with FIG. 3. The processor 160 sends pixelized image data to an output display which includes, for example, images of coronal and sagittal cross-sections, shown respectively as 172 and 174 in FIG. 1.

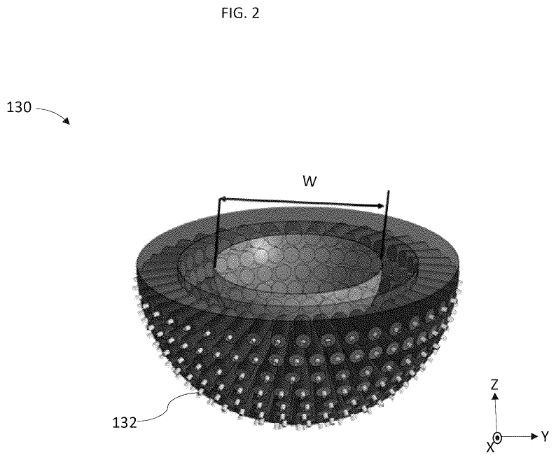

FIG. 2 shows a perspective drawing of the conformal antenna array 130, with its antenna elements distributed in a hemispherical pattern. The inner diameter W of array 130 is approximately equal to the outer diameter of the matching dielectric layer 120. The total number of antenna elements in array 130 is denoted by N_{A}. In an exemplary embodiment of the invention, N_{A} is equal to 96 and W is equal to 210 millimeters (mm). Each antenna element has a coaxial connector 132 and is configured either to transmit or to receive a microwave signal during a given time interval. The colors in FIG. 2 distinguish between elements located at different latitude (or zenith) angles on array 130.

FIG. 3 shows a block diagram of an exemplary microwave imaging method 300, according to the invention. The method consists of the following steps:
Block 310: transmitting ultra-wideband (UWB) microwave signals into a breast;
Block 320: receiving signals in a conformal antenna array;
Block 330: applying frequency-to-time delay pre-processing;
Block 340: applying a synthesized focusing (SF) algorithm;
Block 350: applying a specialized clutter rejection (SCR) algorithm; and
Block 360: generating one or more output images for display.

In block 310, the presence of the matching dielectric layer 120 is important for reducing back reflection at the skin interface and enabling better penetration of the microwave energy into the breast tissue.

In block 320, the amplitude and phase of the received multi-static signals include dominant contributions generated by reflection from the inhomogeneous breast tissues of interest, as well as spurious contributions due to receiver thermal noise and “clutter”. Clutter may arise from many sources, such as multiple scattering effects, diffraction, mutual interactions between antenna elements of the conformal antenna array, and artifacts caused by reflections from the surrounding apparatus.

The pre-processing in block 330 may be implemented, for example, by a Fourier transform, by a finite impulse response (FIR) digital filter, or by using specialized hardware, such as a surface acoustic wave (SAW) device. Such transforms, filters and devices are familiar to those skilled in the art of radar engineering.

The synthesized focusing (SF) algorithm in block 340 is a significant improvement over the “delay and sum” standard focusing method, which is familiar to those skilled in the art of tomography. The theoretical basis for synthesized focusing is briefly summarized below. An in-depth treatment, which includes variants such as inverse, two-step, group, and double focusing, may be found in chapters 2 and 3 of the textbook edited by N. Blaunstein and V. Yakubov, Electromagnetic and Acoustic Wave Tomography: Direct and Inverse Problems in Practical Applications, CRC Press, Taylor and Frances Group, Boca Raton, Fl, USA, 2019. The disclosure of the abovementioned chapters is incorporated by reference in its entirety herein.

In the synthesized focusing algorithm of the invention, all received signals are summed with a time delay calculated for a given focus point, denoted by the vector r_{F}=(x_{F}, y_{F}, z_{F}). The summation includes a large number of angular locations {(φₙ, θₘ), n=1 to N, m=1 to M}, where φₙ and θₘ denote azimuth and elevation (or zenith) angles in the hemispherical pattern of the conformal antenna array 130. Specifically, the summed signal is:

$$U ( {r}_{F} ) = {\sum }_{n} {\sum }_{m} S ( {φ}_{n} , {θ}_{m} , B , {τ}_{n , m} ) \qquad ( equation 1 )$$

Here S is a complex received signal (phase and amplitude), and B is the geometrical radius of a hemispherical surface passing through the phase centers of the conformal antenna array 130. The focusing time delay τ_{n,m} is given by: τ_{n,m}=(2√ε/c)|r_{F}−r_{n,m}|, where c is the speed of light in vacuum, ε is an average value of the dielectric constant of the breast tissue, and r_{n,m}=(B sin θₘ cos φₙ, B sin θₘ sin φₙ, B cos θₘ).

For good spatial resolution, e.g. on the order of 1 to 3 mm., the total number of angular locations, (M×N), is typically between 800 and 1500. The use of a larger or smaller number of locations incurs a penalty of an increase in processing time or a decrease in spatial resolution, respectively.

The specialized clutter rejection (SCR) algorithm in block 350, according to the invention, is a significant improvement over the standard clutter rejection algorithm used in conventional tomography.

In the standard clutter rejection algorithm, the signal after rejection, U, is given by:

$$U ( {r}_{F} ) = {\sum }_{n} ( S ( {r}_{n} , {τ}_{n} ) - {P}_{f ( n )} ) , \qquad equation ( 2 )$$

$${P}_{i} = \frac{1}{N} {\sum }_{n} S ( {r}_{n} , {t}_{i} ) , f ( n ) = [ \frac{{τ}_{n}}{Δ t} ] .$$

In the above equation, tᵢ denotes a sampling time step, Δt is the sampling time increment, and τₙ is a time delay. The average over “n” in the expression for Pᵢ is an average over azimuth angles, φₙ. The brackets in the expression for f(n) indicate integer truncation. The constant term P_{f(n)}, which is subtracted from the signal S, is intended to remove spurious clutter contributions to the received signal. The use of a constant term P_{f(n)} for the clutter contribution presumes that, at each location in azimuth, the clutter has the same temporal form. While this may be adequate for removing clutter associated with re-reflections between the antenna elements of an array having cylindrical symmetry, it does not correctly remove clutter caused by reflections that lack such symmetry, such as artifacts caused by reflection from the surrounding apparatus.

In the specialized clutter rejection (SCR) algorithm of block 350, the signal after clutter rejection, U, is given by:

$${U ( {r}_{F} ) = {\sum }_{n} {\sum }_{m} ( S ( {φ}_{n} , {θ}_{m} , B , {τ}_{n , m} ) - Q \vert}_{m , f ( n )} ) , \qquad ( equation 3 )$$

$${Q}_{m , i} = \frac{1}{N} {\sum }_{n} S ( {φ}_{n} , {θ}_{m} , B , {t}_{i} ) .$$

The constant term Q_{m, f(n)} in the above equation takes on different values for different elevation angles θₘ, and, in this way, rejects clutter both with and without cylindrical symmetry. As we shall see in the Example section below, the use of equation 3 (in place of equation 2) enables the SCR algorithm of the invention to provide images with far better resolution and discrimination between healthy tissue and tumors.

In block 360 of the method of the invention, the digital image processor 160 prepares images for the output display 370. The output images may take the form of several cross-sectional images, such as the coronal and sagittal cross-sections shown as 172 and 174 in FIG. 1.

For real-time mammographic screening by medical personnel, it is highly desirable that the execution of the algorithms in the method of the invention will require no more than, say, 20 seconds. Such short execution times may be achieved by using a MIMO architecture with electronic channel switching in the conformal antenna array 130, and by using parallel processing in the digital image processor 160.

### Example Using a Heterogeneous Breast Phantom

In this example, a heterogeneous breast phantom is prepared in order to simulate the complex nature of real breast tissue, and a protype microwave apparatus is used to generate microwave measurement data. The method of the invention is then used to analyze the measurement data and to prepare output images which demonstrate the high spatial resolution and signal-to-clutter rejection ratio achieved by the invention.

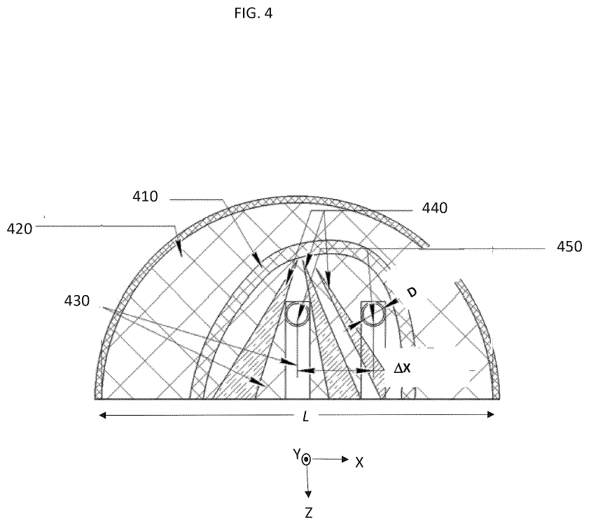

FIG. 4 shows a cross-sectional diagram of the heterogeneous breast phantom and the surrounding matching dielectric layer 120. The skin 410, adipose (fat) tissue 420, and glandular tissue 430 of the breast phantom are made of various ratios of polyurethane mixed with graphite powder. The tumor material 440 is simulated by two plastic balls filled with physiological saline. The ball diameter D is 10 mm., the separation distance ΔX between the balls is 45 mm., and the outer diameter L of the dielectric matching layer 120 is 210 mm.

The following table shows a comparison between the relative permittivity of actual breast samples as reported by Lazebnik and those of the heterogeneous breast phantom, as measured by a coaxial cell connected to a vector circuit analyzer, at a frequency of 4 GHz.

TABLE 1
Relative permittivity values (ε′/ε₀) at 4 GHz

| | Lazebnik | Phantom |
|---|---|---|
| Tissue | (measured) | (measured) |
| skin (410) | 37 | 30 |
| adipose tissue (420) | 5 | 4.5 |
| glandular tissue (430) | 51 | 48 |
| tumor (440) | 68 | 74 |

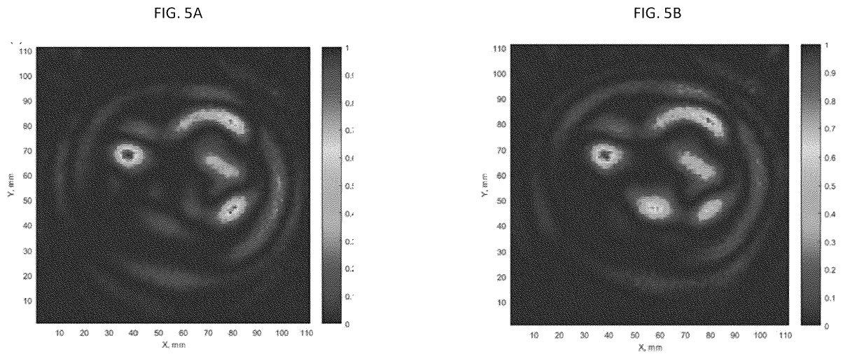

FIG. 5A shows an exemplary coronal cross-sectional image of the heterogeneous breast phantom, obtained using the specialized clutter rejection algorithm of the invention. FIG. 5B shows an analogous image obtained using a standard clutter rejection algorithm. Note the enhanced resolution (of about 5-7 mm.) of the two tumors in FIG. 5A and also the presence of fewer artifacts in FIG. 5A, as compared with FIG. 5B.

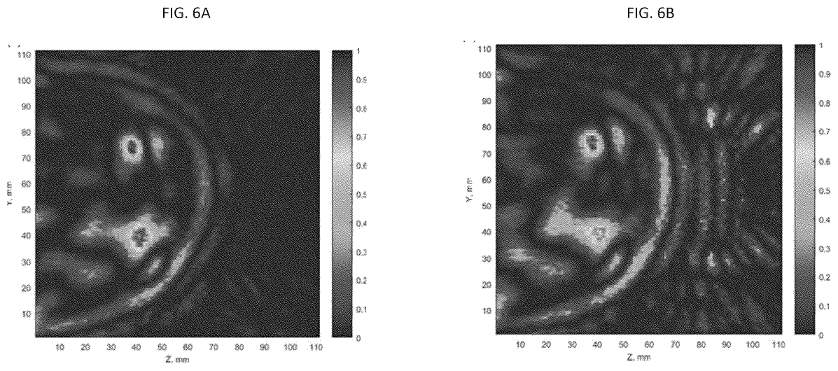

Similarly, FIG. 6A shows an exemplary sagittal cross-sectional image of the heterogeneous breast phantom, obtained using the specialized clutter rejection algorithm of the invention. FIG. 6B shows an analogous image obtained using a standard clutter rejection algorithm. Again, note the enhanced resolution (of about 5-7 mm.) of the two tumors in FIG. 6A and also the presence of fewer artifacts in FIG. 6A, as compared with FIG. 6B.

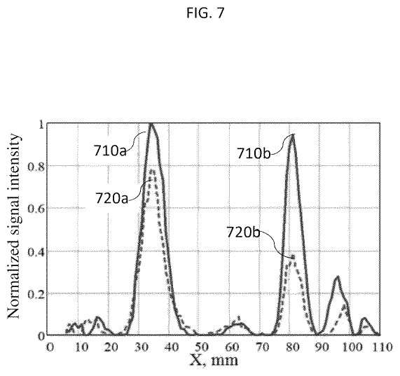

FIG. 7 shows a graph of normalized signal intensity vs. distance along the X-direction inside the heterogeneous breast phantom. The blue curve represents a slice taken from image 5A, obtained using the specialized clutter rejection algorithm of the invention, and the red curve represents an analogous slice taken from image 5B, obtained using a standard clutter rejection algorithm. The peak amplitudes 710a and 710b of the blue curve correspond to the signal intensities of the two tumors which are separated by 45 mm. in the X-direction, as do the peak amplitudes 720a and 720b of the red curve. Note however that the intensity ratio between peaks 710a and 720a is roughly 1.0:0.75, and the intensity ratio between peaks 710b and 720b is roughly 0.9:0.4. These ratios represent a quantitative measure of the improvement in signal-to-clutter ratios achieved with the specialized clutter rejection algorithm of the invention as compared with standard algorithms.

All publications, patents and patent applications mentioned in this specification are herein incorporated in their entirety by reference into the specification. In addition, citation or identification of any reference in this application shall not be construed as an admission that such reference is available as prior art to the present disclosed subject matter.

Certain features of the disclosed subject matter, which are, for clarity, described in the context of separate embodiments, may also be provided in combination in a single embodiment. Conversely, various features of the disclosed subject matter, which are, for brevity, described in the context of a single embodiment, may also be provided separately or in a suitable sub-combination.

In general, the descriptions of the various embodiments of the present disclosure have been presented for purposes of illustration, but are not intended to be exhaustive or limited to the embodiments disclosed. Many other modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. An apparatus for forming a microwave image for the purpose of detection of breast cancer in a patient, the apparatus comprising:
a matching dielectric layer having a first surface proximal to a breast of the patient and a second surface proximal to a conformal antenna array comprising a multiplicity of antenna elements;
an ultra-wideband (UWB) radar signal generator and an UWB radar receiver which are in electrical communication with the conformal antenna array;
a digital image processor in electrical communication with the radar receiver; and
an output device for displaying image data provided by the digital image processor;
wherein each antenna element of the conformal antenna array is configured to transmit and/or to receive microwave signals over a predetermined range of microwave frequencies.

2. The apparatus of claim 1 wherein the UWB radar signal generator and/or the UWB radar receiver comprise(s) a vector network analyzer (VNA).

3. The apparatus of claim 1 wherein the UWB radar signal generator generates pulsed signals having linear or stepped frequency modulation.

4. The apparatus of claim 1 wherein the UWB radar receiver performs frequency to time delay pre-processing.

5. The apparatus of claim 1 wherein the digital image processor performs a synthesized focusing (SF) algorithm and/or a specialized clutter rejection algorithm (SCR).

6. The apparatus of claim 1 wherein the multiplicity of antenna elements is distributed over a multiplicity of azimuth and elevation angles in a hemispherical pattern.

7. The apparatus of claim 1 wherein the conformal antenna array comprises multiple-input, multiple-output (MIMO) electronic channel switching.

8. The apparatus of claim 1 wherein the multiplicity of antenna elements comprises at least 96 elements.

9. The apparatus of claim 1 wherein a pair of antenna elements, in which one element transmits and the other receives, is activated at a given time.

10. The apparatus of claim 1 wherein the matching dielectric layer is configured to improve penetration of the microwave signals into the breast of the patient.

11. The apparatus of claim 1 wherein the predetermined range of microwave frequencies includes a range of two to eight gigahertz.

12. The apparatus of claim 1 wherein the output device is configured to display three-dimensional images and/or two-dimensional cross-sectional images.

13. The apparatus of claim 1 further comprising an examination table supporting the patient in a prone position, the table having a cutout to allow the breast of the patient to protrude into a space beneath the table, under a force of gravity.

14. A computer usable non-transitory storage medium having computer-executable instructions that perform a method of imaging a breast of a patient from microwave measurement data, the method comprising the following steps:
(a) transmitting ultra-wideband (UWB) microwave signals;
(b) receiving signals in a conformal antenna array;
(c) applying frequency-to-time delay pre-processing;
(d) applying a synthesized focusing (SF) algorithm; and
(e) applying a specialized clutter rejection (SCR) algorithm.

15. The method of claim 14 further comprising generating one or more output images for display.

16. The method of claim 15 wherein the one or more output images comprise(s) a coronal and/or a sagittal cross-sectional image.

17. The method of claim 14 wherein the UWB microwave signals propagate through a matching dielectric layer which is proximal to the breast and which is configured to improve penetration of the microwave signals into the breast.

18. The method of claim 14 wherein the conformal antenna array comprises a multiplicity of antenna elements distributed over a multiplicity of azimuth angles and a multiplicity of elevation angles in a hemispherical pattern.

19. The method of claim 18 wherein a total number of angular locations, defined as the product of a number of angles in the multiplicity of azimuth angles and a number of angles in the multiplicity of elevation angles, is between 800 and 1500.

20. The method of claim 14 wherein the frequency-to-time delay pre-processing is performed using a Fourier transform or a finite impulse response (FIR) filter.

21. The method of claim 14 wherein the SF algorithm comprises summation of a signals over a multiplicity of azimuth angles (φₙ) and elevation angles (θₘ), each signal characterized by a focusing time delay (τ_{m,n}).

22. The method of claim 14 wherein the SCR algorithm comprises subtraction of constant values (Q_{m, f(n)}) which may be different for different elevation angles (θₘ).
